(19) United States
(12) Patent Application Publication
(10) Pub. No.: US 2007/0249840 A1
(43) Pub. Date: Oct. 25, 2007

(54) Process for the preparation of bis-benzazolyl compounds

(76) Inventors: Victor Paul Eliu, Lorrach (DE); Julia Hauser, Grenzach-Wyhlen (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/821,009

(22) Filed: Jun. 21, 2007

Related U.S. Application Data
(63) Continuation of application No. 10/399,312, filed on Apr. 16, 2003, now abandoned.

(30) Foreign Application Priority Data
Oct. 18, 2000 (EP) 00810961.3

(51) Int. Cl. C07D 263/64; C07D 403/10; C07D 417/10

(52) U.S. Cl. 548/159; 548/219; 548/305.4

(57) ABSTRACT

The present invention provides a process for the preparation of a compound of the formula 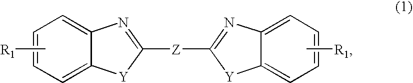 wherein

Y represents —O—, —S— or —N(R₂)—,

R₂ being hydrogen, C₁-C₁₀alkyl or aralkyl; Z represents a 2,5-furanyl, 2,5-thiophenyl, 4,4′-stilbenyl or a 1,2-ethylenyl residue and

R₁ represents hydrogen, halogen, C₁-C₁₀alkyl, C₁-C₁₀alkoxyl, cyano, COOM or SO₃M,
M being hydrogen or an alkaline or alkaline earth metal, characterized by reacting a compound of the formula 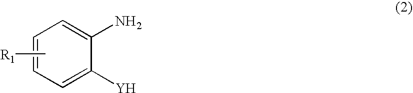 with a dicarboxylic acid of the formula
HOOC-Z-COOH (3)
or an ester thereof, Y, Z and R₁ being as previously defined, in N-methylpyrrolidone or N,N-dimethylacetamide, in the presence of an acidic catalyst and, optionally, in the presence of a secondary solvent capable of removing water from the reaction mixture, which are useful as optical whitening agents for natural and synthetic materials.

This application is a continuation of app. Ser. No. 10/399,312, pending, which is a 371 of international app. No. PCT/EP01/11644, filed Oct. 9, 2001, the disclosures of which are hereby incorporated by reference.

The present invention relates to a process for the preparation of bis-benzazolyl compounds which are useful as optical whitening agents for natural and synthetic materials.

Various methods for the preparation of such compounds are known.

Thus, for example, U.S. Pat. No. 4,508,903 describes the preparation of 4,4′-bis.benzoxazol-, benzthiazol- and benzimidazol-2-ylstilbenes by dimerisation of the corresponding p-chloromethylphenylbenzazoles. However, such methods suffer from the disadvantage that the preparation of the intermediates involves several reaction steps, subsequently rendering poor overall yields.

Of particular practical interest are processes in which dicarboxylic acids or their derivatives are reacted with bifunctional aromatic compounds to form the heterocyclic rings in a single reaction step.

Thus, for example, European Patent 31,296 discloses a process for the preparation of benzoxazolyl and benzimidazolyl compounds by condensation of organic carboxylic acids with o-aminophenols and o-phenylenediamines in a solvent mixture consisting of diphenyl ether and diphenyl in the presence of acidic catalysts. Furthermore, British Patent 1,201,287 describes the preparation of 2,5-bisbenzoxazol-2-yl thiophenes by condensation of thiophene-2,5-dicarboxylic acid with o-aminophenols in, for example, refluxing 1,2,4-trichlorobenzene in the presence of boric acid. Such processes are disadvantageous since they demand extremely high reaction temperatures, resulting in the formation of impurities which are difficult to remove from the final products and, as a consequence, loss of product yields. Furthermore, such high-boiling solvents are also difficult to remove from the reaction products and may further result in crust-formation inside reaction vessels, thus impeding work-up of the final products. Additionally, employment of chlorinated aromatic solvents in the present day is undesirable for ecological reasons.

Surprisingly, a new, advantageous process for the preparation of bis-benzazolyl compounds has now been found, which provides these compounds in high yields of excellent purity under reaction conditions well suited to commercial processes.

Accordingly, the current invention provides a process for the preparation of a compound of the formula

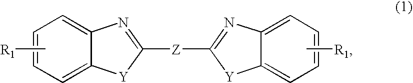

wherein

Y represents —O—, —S— or —N(R₂)—,

R₂ being hydrogen, C₁-C₁₀alkyl or aralkyl;

Z represents a 2,5-furanyl, 2,5-thiophenyl, 4,4′-stilbenyl or a 1,2-ethylenyl residue and

R₁ represents hydrogen, halogen, C₁-C₁₀alkyl, C₁-C₁₀alkoxy, cyano, COOM or SO₃M,

M being hydrogen or an alkaline or alkaline earth metal, characterized by reacting a compound of the formula

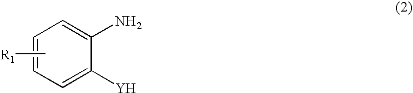

with a dicarboxylic acid of the formula
HOOC-Z-COOH (3)

or an ester thereof, Y, Z and R₁ being as previously defined, in N-methylpyrrolidone or N,N-dimethylacetamide, in the presence of an acidic catalyst and, optionally, in the presence of a secondary solvent capable of removing water from the reaction mixture.

The molar ratios of the compound of formula (2) to the compound of formula (3) may vary over wide ranges. However, it is advantageous to react at least two moles of the compound of formula (2) with at least one mole of the dicarboxylic acid of formula (3). Alternatively, a mono- or diester, preferably a diester, of the compound of formula (3) may be employed. Appropriate esters are those derived from a C₁-C₁₀-, preferably C₁-C₄alcohol, diethyl esters being most preferred.

The process of the invention is particularly suitable for the preparation of a compound of formula (1) in which

Y represents —O—, —S— or —N(R₂)—,

R₂ being hydrogen or C₁-C₄alkyl;

Z is as defined previously and

R₁ represents hydrogen or C₁-C₄alkyl and, more especially for compounds of formula (1) in which

Z represents a 2,5-furanyl or a 2,5-thiophenyl residue and also for those in which Z represents a 4,4′-stilbenyl or a 1,2-ethylenyl residue.

As reaction medium for the process of the invention N-methylpyrrolidone or N,N-dimethylacetamide or mixtures thereof are most preferred. It is also possible to use N-methylpyrrolidone or N,N-dimethylacetamide or mixtures thereof together with a further high boiling inert solvent, e.g. toluene or xylene. The use of N-methylpyrrolidone is especially preferred.

The acidic catalyst employed in the process of the invention may be selected from the group consisting of boric acid, phosphoric acid, titanium C₁-C₄orthoesters or tin derivatives, boric acid or a titanium C₁-C₄orthoester, especially tetrapropyl or tetrabutyl ester, being of preference. The amount of catalyst employed may vary over wide ranges and is dependent on the chemical entity. Thus, for example, amounts varying from 0.01 to 50 mole %, based on the amount of compound (2), preferably 0.1 to 30 mole % may be used.

Reaction of compounds of the formulae (2) and (3) may be carried out within a wide temperature range, but is preferably within the range of between 100 and 250° C., in particular within a temperature range of between 150 and 200° C.

The presence of a secondary solvent is of particular importance when the compound of formula (3) is in the form of a monoester or, especially, the free dicarboxylic acid. In these cases, water formed during the course of the reaction may be continuously removed from the reaction mixture. Examples of suitable solvents, without the choice being limited thereto, are selected from the group consisting of toluene, the xylenes and isomeric mixtures thereof and pyridine, toluene and xylene being especially effective.

The reaction of the invention is normally carried out under atmospheric pressure. However, under certain circumstances, it may prove advantageous to perform the reaction under higher or lower pressures.

Within the scope of the compounds of formulae (1) and (2), when R₁ represents halogen this may be fluorine, bromine, iodine or, especially, chlorine.

C₁-C₁₀alkyl groups R₁ and/or R₂ may be branched or unbranched such as methyl, ethyl, n-propyl, isopropyl, n-butyl, sec-butyl, isobutyl, t-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, n-nonyl or n-decyl. C₁-C₁₀alkyl esters of compound of formula (3) are substituted correspondingly.

C₁-C₁₀alkoxy groups R₁ may be branched or unbranched such as methoxy, ethoxy, n-propoxy, isopropoxy, n-butoxy, sec-butoxy, isobutoxy, t-butoxy, 2-ethylbutoxy, n-pentoxy, isopentoxy, 1-methylpentoxy, 1,3-dimethylbutoxy, n-hexoxy, 1-methylhexoxy, n-heptoxy, isoheptoxy, 1,1,3,3-tetramethylbutoxy, 1-methylheptoxy, 3-methylheptoxy, n-octoxy, 2-ethylhexoxy, 1,1,3-trimethylhexoxy, 1,1,3,3-tetramethylpentoxy, n-nonoxy or n-decoxy.

Aralkyl groups R₂ may be benzyl or phenethyl which may be substituted by halogen, C₁-C₁₀alkyl or C₁-C₁₀alkoxy groups or, preferably, unsubstituted.

The alkaline or alkaline earth metal M may be selected from the group consisting of K, Na, Ca or Mg, but is preferably K or Na.

The following Examples further illustrate the present invention, without intending to be restrictive thereto:

### EXAMPLE 1

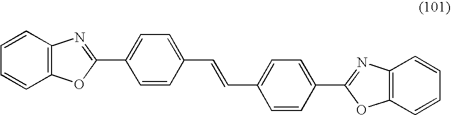

250 g of N-methylpyrrolidone are charged to a reaction vessel and 82 g of 98% stilbene-4,4′-dicarboxylic acid, followed by 75 g of 99% 2-aminophenol, 10 g of boric acid and 30 g of xylene are added with stirring. The apparatus, equipped with a Dean and Stark water trap, is evacuated and the vacuum released with nitrogen. The light yellow suspension is heated to 195° C. and stirred at this temperature for 18 hours, during which time 23-25 ml of water and approximately 25 g of xylene are distilled off through the water trap. The reaction mixture is cooled to 20° C. and stirring continued for 1 hour at this temperature. The yellow suspension is filtered, washed with 100 g of N-methylpyrrolidone to give 350 g of a brown solution which may be used as solvent for a further charge and then with three 80 g portions of water. The resulting press-cake is dried under a vacuum of 50 mbar at 100° C. to yield 120 g of the compound of formula (101) as a yellow solid, characterized by a UV absorption maximum λₘₐₓ at 368 nm with an extinction coefficient ε of 71000.

### EXAMPLE 2

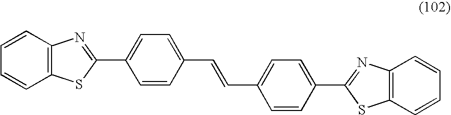

By following the procedure described in Example 1, but replacing the 2-aminophenol by 82 g of 98% 2-thiophenol and the boric acid by 3 g of titanic acid tetra-isopropyl ester, there are obtained 115 g of the compound of formula (102) as a yellow solid, characterized by a UV absorption maximum λₘₐₓ at 375 nm with an extinction coefficient ε of 62000 and by the following ¹H-NMR data in D₆-DMSO:

8.12, 4H, m; 8.00, 6H, m; 7.85, 4H, m and 7.48, 4H, m.

### EXAMPLE 3

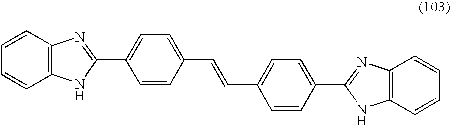

By following the procedure described in Example 1, but replacing the 2-aminophenol by 72 g of 99% 1,2-phenylenediamine, there are obtained 110 g of the compound of formula (103) as a yellow solid, characterized by a UV absorption maximum λₘₐₓ at 370 nm with an extinction coefficient ε of 63000 and by the following ¹H-NMR data in D₆-DMSO:

13.0, 2H, s; 8.22, 4H, d, j=7 Hz; 7.80, 4H, d, j=7 Hz; 7.68, 2H, d, j=7 Hz; 7.54, 2H, d, j=7 Hz; 7.48, 2H, s and 7.22, 4H, t, j=7 Hz.

### EXAMPLE 4

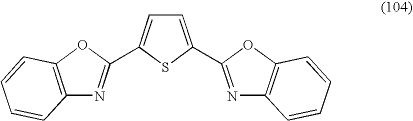

200 g of N-methylpyrrolidone are charged to a reaction vessel and 52 g of 98% thiophene-2,5-dicarboxylic acid, followed by 72 g of 99% 2-aminophenol, 10 g of boric acid and 30 g of toluene are added with stirring. The apparatus, equipped with a Dean and Stark water trap, is evacuated and the vacuum released with nitrogen. The light yellow suspension is heated to 185° C. and stirred at this temperature for 12 hours, during which time 23-25 ml of water and approximately 25 g of toluene are distilled off through the water trap. The reaction mixture is cooled to 20° C. and stirring continued for 1 hour at this temperature. The yellow suspension is filtered, washed with 10 g of N-methylpyrrolidone to give 300 g of a brown solution which may be used as solvent for a further charge and then with three 80 g portions of water. The resulting press-cake is dried under a vacuum of 50 mbar at 100° C. to yield 75 g of the compound of formula (104) as a yellow solid, characterized by a UV absorption maximum λₘₐₓ at 372 nm with an extinction coefficient ε of 52000 and by the following ¹H-NMR data in D₆-DMSO:

8.10, 2H, s; 7.82, 4H, m and 7.50, 4H, m.

### EXAMPLE 5

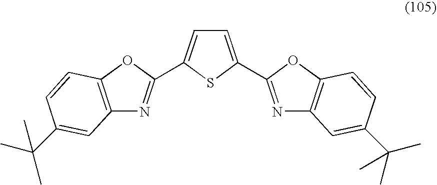

By following the procedure described in Example 4, but replacing the 2-aminophenol by 110 g of 2-amino-4-t-butylphenol, the boric acid by 2.2 g of isopropyl-ortho-titanate and the toluene by 30 g of xylene, there are obtained 125 g of the compound of formula (105) as a yellow solid, characterized by a UV absorption maximum λₘₐₓ at 375 nm with an extinction coefficient ε of 51000 and by a singlet at 1.30 ppm in the ¹H-NMR spectrum in D₆-DMSO.

### EXAMPLE 6

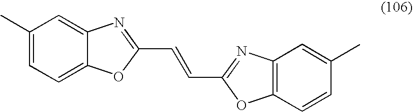

200 g of N,N-dimethylacetamide are charged to a reaction vessel and 35 g of 98% fumaric acid, followed by 82 g of 2amino-4-methylphenol, 10 g of boric acid and 30 g of xylene are added with stirring. The apparatus, equipped with a Dean and Stark water trap, is evacuated and the vacuum released with nitrogen. The light yellow suspension is heated to 160° C. and stirred at this temperature for 10 hours, during which time 23-25 ml of water and approximately 25 g of xylene are distilled off through the water trap. The reaction mixture is cooled to 20° C. and stirring continued for 1 hour at this temperature. The yellow suspension is filtered, washed with 100 g of N,N-dimethylacetamide and then with three 80 g portions of water. The resulting press-cake is dried under a vacuum of 50 mbar at 100° C. to yield 85 g of the compound of formula (106) as a yellow solid, characterized by a UV absorption maximum λₘₐₓ at 365 nm with an extinction coefficient ε of 42000.

### EXAMPLE 7

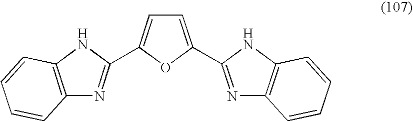

200 g of N-methylpyrrolidone are charged to a reaction vessel and 65 g of furan-2,5-dicarboxylic acid, followed by 72 g of 99% 1,2-phenylenediamine and 10 g of boric acid are added with stirring. The apparatus, equipped with a Dean and Stark water trap, is evacuated and the vacuum released with nitrogen. The light yellow suspension is heated to 175° C. and stirred at this temperature for 12 hours, during which time 28 g of ethanol are distilled off under a weak vacuum. The resulting solution is cooled to 20° C. and stirring continued for 1 hour at this temperature. The yellow suspension is filtered, washed with 100 g of N-methylpyrrolidone to give 300 g of a brown solution which may be used as solvent for a further charge and then with three 80 g portions of water. The resulting press-cake is dried under a vacuum of 50 mbar at 100° C. to yield 95 g of the compound of formula (107) as a yellow solid, characterized by a UV absorption maximum λₘₐₓ at 375 nm with an extinction coefficient ε of 42000.

1. A process for the preparation of a compound of the formula 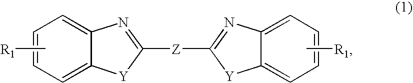
wherein
Y represents —O—, —S— or —N(R₂)—,
R₂ being hydrogen, C₁-C₁₀alkyl or aralkyl;
Z represents a 2,5-furanyl, 2,5-thiophenyl, 4,4′-stilbenyl or a 1,2-ethylenyl residue and
R₁ represents hydrogen, halogen, C₁-C₁₀ alkyl, C₁-C₁₀alkoxyl, cyano, COOM or SO₃M,
M being hydrogen or an alkaline or alkaline earth metal,
which comprises reacting a compound of the formula 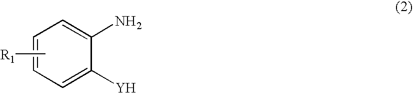
with a dicarboxylic acid of the formula
HOOC-Z-COOH (3)
or an ester thereof,
Y, Z and R₁ being as previously defined,
in a solvent consisting of N-methylpyrrolidone or N,N-dimethylacetamide, or in a solvent mixture of N-methylpyrrolidone or N,N-dimethylacetamide and a secondary solvent capable of removing water from the reaction mixture, which secondary solvent is present up to 15% by weight, based on the weight of N-methylpyrrolidone or N,N-dimethylacetamide,
and in the presence of an acidic catalyst.

2. A process according to claim 1, in which at least two moles of the compound of formula (2) are reacted with at least one mole of the dicarboxylic acid of formula (3) or an ester thereof.

3. A process according to claim 1 for the preparation of a compound of formula (1) in which
Y represents —O—, —S— or —N(R₂)—,
R₂ being hydrogen or C₁-C₄alkyl;
Z is as defined in claim 1 and
R₁ represents hydrogen or C₁-C₄alkyl.

4. A process according to claim 3 in which Z represents a 2,5-furanyl or a 2,5-thiophenyl residue.

5. A process according to claim 3 in which Z represents a 4,4′-stilbenyl or a 1,2-ethylenyl residue.

6. A process according to claim 1 in which reaction of compounds of formulae (2) and (3) is carried out in N-methylpyrrolidone.

7. A process according to claim 1 in which the acidic catalyst is selected from the group consisting of boric acid, phosphoric acid, titanium C₁-C₄orthoesters and tin derivatives.

8. A process according to claim 7 in which the acidic catalyst is boric acid or a titanium C₁-C₄orthoester.

9. A process according to claim 1 in which reaction of compounds of the formulae (2) and (3) is carried out within a temperature range of between 100 and 250° C.

10. A process according to claim 6 in which reaction of compounds of the formulae (2) and (3) is carried out within a temperature range of between 150 and 200° C.

11. A process according to claim 1 in which the secondary solvent capable of removing water from the reaction mixture is selected from the group consisting of toluene, the xylenes, isomeric mixtures of xylenes and pyridine.
